(19) United States
(12) Patent Application Publication
(10) Pub. No.: US 2005/0054994 A1
(43) Pub. Date: Mar. 10, 2005

(54) Catheters with suction capability and related methods and systems for obtaining biosamples in vivo

(76) Inventors: Iulian Cioanta, Weston, FL (US); Richard Barry Klein, Cary, NC (US); Jacob Lazarovitz, Hod Hasharon (IL)

Correspondence Address: GANZ LAW, P.C., P O BOX 2200, HILLSBORO, OR, 97123, US

(21) Appl. No.: 10/827,741

(22) Filed: Apr. 19, 2004

(30) Foreign Application Priority Data
Sep. 25, 2002 (WO) PCT/US02/30354

(51) Int. Cl. A61B 17/20; A61M 1/00

(52) U.S. Cl. 604/317

(57) ABSTRACT

Methods, systems, and computer program products for obtaining a sample in vivo and/or treating a subject include positioning an elongated transurethral catheter in the prostatic urethra of a subject, the catheter having a bladder anchoring balloon, at least one biosample entry port disposed axially away from the bladder anchoring balloon, and an axially extending biosample flow channel in fluid communication with the biosample entry port held internally in the catheter. The anchoring balloon is inflated to position the catheter so that the fluid entry port is proximate the prostatic urethra of the subject and prostatic fluid is suctioned from the prostatic urethra into the biosample entry port and into the biosample flow channel. The catheter can include a thermal treatment balloon and/or dilatation balloon and the suctioned sample can be obtained concurrently with, during, or proximate in time to the applied treatment. The catheter can be configured to allow urine to drain therethrough during the treatment/collection of the sample while keeping the urine isolated from the collected prostatic fluid sample.

### RELATED APPLICATIONS

This application claims priority from U.S. Provisional Application Serial No. 60/330,029, filed Oct. 17, 2001, and PCT Application Serial No. PCT/JUS02/30354, filed Sep. 25, 2002, the contents of which are hereby incorporated by reference as if recited in full herein.

### FIELD OF THE INVENTION

The present invention relates to catheters configured for insertion into a lumen or body cavity of a subject, and is particularly suitable for catheters configured for insertion into the male urethra.

### BACKGROUND OF THE INVENTION

Conventionally, several types of thermal treatment systems have been proposed to treat certain pathologic conditions of the body by heating or thermally ablating targeted tissue. These thermal treatment systems have used various heating sources to generate the heat necessary to treat or ablate tile targeted tissue. For example, laser, microwave, and radio-frequency (RF) energy sources have been proposed to produce heat that is then directed to the targeted tissue in or around the selected body cavity. Thermal treatment systems have been used to thermally ablate prostate tissue as well as to thermally treat or ablate the tissue of other organs, body cavities, and/or natural lumens. Other proposed treatments include balloon dilatation applied internally without the concurrent application of heat.

One particularly successful thermal ablation system ablates the prostate by a thermocoagulation process. This thermal ablation system employs a closed loop liquid or water-induced thermotherapy system that heats liquid, typically water, external to the body and then directs the circulating heated water into a treatment catheter. The treatment catheter is inserted through the penile meatus and held in position in the subject prior to initiation of the treatment to expose localized tissue in the prostate to ablation temperatures. The treatment catheter includes an upper end portion which, in operation, is anchored against the bladder neck and an inflatable treatment segment which is held relative to the anchored upper end portion such that it resides along the desired treatment region of the prostate. In operation, the treatment segment expands, in response to the captured circulating fluid traveling therethrough, to press against the targeted tissue in the prostate and to expose the tissue to increased temperatures associated with the circulating liquid, thereby thermally ablating the localized tissue at the treatment site. In addition, the pressurized contact can reduce the heat sink effect attributed to blood circulation in the body, thus enhancing the depth penetration of the heat transmitted by the inflatable treatment segment into the prostatic tissue.

As an acceptable alternative to surgery (transurethral resection of the prostate (TURP)), the use of water-induced thermotherapy has been shown to be a successful and generally minimally invasive treatment of BPH (benign prostatic hyperplasia). Generally stated, the term “BPH” refers to a condition wherein the prostate gland enlarges and the prostatic tissue increases in density that can, unfortunately, tend to close off the urinary drainage path. This condition typically occurs in men as they age due to the physiological changes of the prostatic tissue (and bladder muscles) over time. To enlarge the opening in the prostate urethra (without requiring surgical incision and removal of tissue), the circulating hot water is directed through the treatment catheter which is inserted into the penile meatus up through the penile urethra and into the prostate as described above. The treatment segment expands with the hot water held therein to press the inflated treatment segment against the prostate, which then conductively heats and thermally ablates the prostatic tissue. The circulating water is typically heated to a temperature of about 60°-62° C. and the targeted tissue is thermally treated for a period of about 45 minutes to locally kill the tissue proximate the urinary drainage passage in the prostate and thereby enlarge the urinary passage through the prostate.

It is believed that chronic prostatitis is one of the most common reasons why men visit urologists, even being characterized as the condition responsible for more outpatient visits than benign prostatic hyperplasia (“BPH”) or prostate cancer. At least one report states that 35-50% of men will be affected by prostatitis at some time in their lives. The treatments conventionally used to treat this condition have been generally problematic; most of the treatments have provided little hope that the condition can be predictably treated in a manner which could successfully alleviate the pain experienced by a large percentage of these individuals. Indeed, prostatitis has been termed “a waste basket of clinical ignorance” because of the lack of knowledge about the basic epidemiology of the disease and also the diagnosis and treatments available for same. See McNaughton Collins et al., How Common is Prostatitis? A National Survey of Physician Visits, Jnl. of Urology, Vol. 159, pp. 1224-1228 (April 1998).

Unlike BPH, which occurs primarily in older men, prostatitis can occur in both younger (men in age groups of 18-50 (or younger)) and older men (over the age of 50), with the median reported patient age at about 40 years of age. See id. at 1228. It is thought to be the most common urologic diagnosis for men less than 50 years of age.

There are several classifications or types of prostatitis, each of which may have different characteristics, manifestations, symptoms, or treatment protocols. These are Type I (acute bacterial prostatitis), Type II (chronic bacterial prostatitis), Type III chronic (non-bacterial) prostatitis and/or chronic pelvic pain syndrome (CPPS), and Type IV (asymptomatic inflammatory prostatitis). See Nickel et al., Research Guidelines for Chronic Prostatitis: Consensus Report From the First National Institutes of Health International Prostatitis Collaborative Network, Urology, 54(2), pp. 229-233, 230 (1999).

The Type III prostatitis class (non-bacterial chronic prostatitis) is generally associated with urogenital pain in the absence of uropathogenic bacteria detected by standard microbiological methodology. See Nickel et al., Research Guidelines for Chronic, supra, p. 230. Type III prostatitis may be further defined as IIIA (inflammatory) or IIIB (noninflammatory). The IIIA inflammatory type prostatitis can be identified based on the presence of leukocytes in expressed prostatic secretions or fluids, post prostatic massage urine, or semen, while the IIIB non-inflammatory type can be identified based on the absence of detectable leukocytes in similar specimens. This type of prostatitis may also be associated with variable voiding, sexual dysfunction, and/or psychologic alterations (particularly depression).

Only a small number of reported prostatitis cases are believed to be of the Type I or acute bacterial type, while the remaining classes of chronic prostatitis may affect an estimated 30 million men in the United States. In any event, as noted above, one of the primary symptoms of prostatitis is a chronic urogenital pain that can negatively impact the quality of life of individuals experiencing this condition. This pain may occur with urination, ejaculation, or in other urogential manifestations. It has been stated that the impact on the quality of life may be similar to those patients suffering unstable angina, a recent myocardial infarct, or active Crohn's disease. As such, chronic prostatitis is a major health care issue. See J. Curtis Nickel, Prostatitis: Myth and Realities, Urology 51 (3), pp. 362-366 (1998).

Recently proposed treatments for prostatitis include internally massaging the prostate during the application of thermal treatment. See co-pending and co-assigned Provisional U.S. Patent Application Ser. No. 60/308,344, the contents of which are hereby incorporated by reference as if recited in full herein.

Notwithstanding the above, there remains a need to provide improved methods and devices for treating diseases of the urethra or prostate including one or more of BPH, chronic prostatitis, and cancer.

### SUMMARY OF THE INVENTION

The present invention provides catheters and related systems, methods, and computer program products that can capture biofluids and/or biosamples in vivo, and/or that may enhance the treatment of certain diseases of the body when the catheter is used both to apply a dilatation and/or thermal therapy to a targeted region in a cavity or lumen of the subject. The present invention may be particularly suitable for collecting prostatic fluid samples and/or for treating diseases of the prostate such as BPH, prostatitis, and cancer.

In certain embodiments, the catheter can be configured to administer a thermal therapy to the targeted region. In particular embodiments, the thermal therapy can be applied with an internal massage. In other embodiments, the collecting or capturing of the biofluids can comprise suctioning the biosamples. The collecting may be carried out continuously, semi-continuously, or at selected times over the course of the treatment. The concurrent combination of pressure and/or heat (thermal with an internal massage therapy) with suctioning may provide increased therapeutic responsiveness over massage and/or thermotherapies alone.

In embodiments for treating diseases of the prostate, drawing biofluid from the prostate may enhance the efficacy of the treatment and/or can provide prostatic fluid specimens that are substantially void of urine. In addition, the quantity of biofluid released over the treatment period can be monitored and analyzed (flow rate/volume etc.) to evaluate the treatment delivered with internal tissue activity in the body.

In certain embodiments, the thermal treatment period can extend from about 5 min to about 90 min (or longer). In particular embodiments, the thermal therapy can be administered by employing circulating heated fluid in the catheter. As such, the fluid can be heated, but controlled, so that the prostatic temperature is exposed to predetermined temperatures for selected time periods. The duration of the treatment may not include the initial time to reach the desired treatment temperature (or the time to decrease therefrom post-treatment).

In addition, the catheter can be used to capture a biosample of the prostatic fluid to monitor the efficacy of a therapeutic agent or treatment regimen. This may allow easier identification of elevated or decreased levels (or the presence or absence) of an analyte(s) of interest. In addition, the amount of biofluid collected over a particular period may be indicative of a disease state, condition, impairment in function of the prostate. The catheters and methods provided by certain embodiments of the present invention can capture the biofluid specimen in a manner that provides the specimen ex vivo in substantially the same condition (concentration/constituents) as it was in vivo at entry into the catheter.

In addition, the collection of the biosample can be performed such that it is obtained concurrently with, after, or before a radiation treatment (or chemotherapy) to evaluate alterations in quantity or content of the collected biospecimen.

Certain embodiments of the present invention are directed to methods for obtaining a sample and/or treating a subject. The method includes: (a) positioning an elongated transurethral catheter in the prostatic urethra of a subject, the catheter having a bladder anchoring balloon, at least one biosample entry port disposed axially away from the bladder anchoring balloon, and an axially extending biosample flow channel in fluid communication with the biosample entry port held internally in the catheter; (b) inflating the anchoring balloon to position the catheter so that the biosample entry port is proximate the prostatic or membraneous urethra of the subject; and (c) suctioning prostatic fluid from the prostatic urethra into the biosample entry port and into the biosample flow channel.

In particular embodiments, the method can be carried out so as to direct the prostatic fluid to exit the body so that the suctioned prostatic fluid is substantially void of urine and then capturing the prostatic fluid after it exits the body. As such, the method may include monitoring the quantity or flow rate of the captured fluid over a predetermined time. The catheter may be configured to capture prostatic fluid at the acini region of the prostate, the fluid exiting this region may be enhanced by temporal thermal internal massage therapy.

Other embodiments are directed at methods for treating the prostate of a subject. The operations of the method can include: (a) inserting a catheter into the urethra of a subject, the catheter having, in serial order from the most distal portion, a bladder anchoring balloon, at least one expandable treatment balloon, and at least one fluid entry port formed in the wall of the catheter, the catheter also having an axially extending biosample flow path in fluid communication with the fluid entry port disposed internal of the catheter wall; (b) expanding the bladder anchoring balloon to contact and reside against the bladder neck of the subject to secure the catheter in position in the subject; (c) heating fluid to a desired temperature; (d) directing heated fluid to travel captured in the catheter to the at least one expandable treatment balloon; (e) inflating the at least one treatment balloon responsive to the directing step, wherein, in position, the inflated treatment balloon takes on a radially expanded configuration and circumferentially contacts targeted tissue in the prostatic urethra; (f) heating a targeted region in the prostatic urethra to a temperature of between about 40-67° C. for a desired treatment time of at least about 20 minutes; and (g) drawing a biosample comprising prostatic fluid into the fluid entry port and into the biosample flow path of the catheter.

In particular embodiments, the suctioning step is carried out at least intermittently during the heating step. In other embodiments, the suctioning step is carried out substantially continuously during the heating step while in still other embodiments the suctioning step is carried out at a plurality of discrete intervals for a predetermined period of time during the treatment.

Still other embodiments are directed to methods of collecting a biosample in a subject. The method includes: (a) inserting a catheter into the male urethra, the catheter having a biofluid travel path defined therein; (b) collecting a biosample from the prostatic urethra into the catheter in vivo; and (c) directing the collected biosample to travel in the biofluid travel path and to exit the body in a manner that keeps the biosample substantially void of urine.

In particular embodiments, the collecting step can suction the biosample and the method can also include the steps of applying heat to the prostatic urethra proximate in time to or during the collecting step and/or allowing urine to drain from the bladder of the subject during the collecting or suctioning step.

Similarly, other embodiments are directed to methods of collecting a biosample in a subject that is substantially void of urine. The operations include: (a) inserting a catheter into a urethra of a subject, the catheter having a biofluid travel path defined therein; (b) suctioning a biosample from a targeted location along the urethra into the catheter in vivo; and (c) directing the suctioned biosample to travel in the biofluid travel path and to exit the body in a manner that keeps the biosample substantially void of urine. The urethra can be either the female or male urethra.

Still other embodiments are directed to sets of prostatic treatment catheters having expandable treatment balloons. The treatment balloons are configured on a flexible catheter sized and configured to be inserted into the male urethra. The treatment balloons are sized in about 0.5 cm increments from about 1 cm to 6 cm such that, a clinician can select one of the catheters having the desired length treatment balloon. The treatment balloon having a length so that in position in the body it resides above the verumontanum of the subject in the prostatic urethra. Each of the catheters also comprise a plurality of prostate drainage ports in communication with a prostate drainage lumen held internal of the catheter.

An additional embodiment of the present invention is directed toward a system for collecting a prostatic fluid specimen in vivo in a subject. The system includes a transurethral elongated catheter having an outer wall. The catheter has at least one prostate drainage port formed through the outer wall and an axially extending prostatic flow channel held therein. The system can include a suction source in fluid communication with the at least one prostate drainage port. In operation, the suction source provides a suction force sufficient to draw prostatic fluid into the at least one prostate drainage port and into the prostatic flow channel to thereby cause the prostatic fluid to flow out of the body of the subject so as to be collected for evaluation.

Other embodiments are directed at systems for collecting a prostatic fluid specimen in vivo in a subject and/or facilitating the administration of a treatment to a subject. The system includes an elongated transurethral catheter having an outer wall. The catheter comprises: (a) a plurality of axially extending internal fluid flow channels disposed in the catheter, an inlet circulating fluid channel, an outlet fluid circulating channel, a urinary drainage channel, and an axially extending prostatic fluid channel, wherein the prostatic fluid channel is in fluid isolation from the urine drainage and inlet and outlet channels; (b) an outwardly expandable treatment or dilatation balloon; (c) a bladder anchoring balloon; and (d) at least one prostate drainage port formed through the outer wall of the catheter in fluid communication with the prostatic fluid channel. The system also includes a quantity of circulating fluid in the inlet and outlet channels; a heater operably associated with fluid traveling in the inlet and outlet channels; a pump operably associated with the circulating fluid to cause the fluid to circulate in the catheter; at least one temperature sensor operably associated with the heater and the circulating fluid in the inlet and outlet channels; and a suction source in fluid communication with the at least one prostate drainage port. In operation, the suction source provides a suction force sufficient to draw prostatic fluid into the at least one prostate drainage port and into the prostatic flow channel to thereby flow out of the body of the subject so as to be able to be collected for evaluation.

In particular embodiments, the pump can be a pulsating pump configured to circulate the fluid in a pulsating flow. In addition, the prostate drainage port can be a plurality of discrete ports spaced on the catheter such that, in position, they primarily reside proximate the verumontanum region.

In other embodiments, the present invention is directed to computer program products for obtaining a biosample of the prostatic urethra. The product includes: (a) computer readable program code for activating and applying a suction force to a fluid channel extending from a suction source located external of the body of the subject to the prostatic urethra via a catheter that is in fluid communication with the prostate; and (b) computer readable program code for drawing in and capturing a biosample comprising prostatic fluid in the catheter. The captured biosample can be held so that it is substantially void of urine as it is directed to exit the subject in the catheter.

Other embodiments are directed to computer program products for administering a thermal therapy to a subject, the thermal treatment being provided by a closed loop system having a heater, a circulating fluid pump, a suction source, and a trans-lumenal catheter configured and sized to be inserted through the male urethra. The catheter including a biosample collection port and channel and an outwardly expandable treatment balloon thereon. The balloon is configured, in operation, to expand while the catheter circulates heated fluid to heat the prostatic urethra via the expandable treatment balloon. The computer program product comprises a computer readable storage medium having computer readable program code embodied in the medium, the computer-readable program code comprising: (a) computer readable program code for controlling the temperature of fluid circulating in the catheter so that the temperature of the fluid entering the catheter to travel to the expandable treatment balloon is between about 40-67° C.; (b) computer readable program code for timing the duration of the thermal massage treatment so that the treatment lasts from about 20 minutes to 1 hour; and (c) computer readable program code for activating the suction source to draw a biosample from the prostatic urethra into the catheter. In particular embodiments, the thermal massage can be described as an internal thermal massage where the treatment balloon repetitively expands and contracts to apply a massage to the prostate.

Particular embodiments of the present invention are directed to methods for treating prostatitis. The method comprises: (a) inserting a catheter with a suction port and associated flow channel and at least one expandable treatment balloon thereon into the urethra of a subject, the treatment balloon positioned to extend outwardly about the perimeter of a portion of the catheter; (b) inflating the at least one treatment balloon, wherein, in position, the inflated treatment balloon takes on a radially expanded configuration and circumferentially contacts targeted tissue in the prostatic urethra; (c) heating a targeted region in the prostatic urethra to a temperature of between about 40-47° C. for a desired treatment time of at least 20 minutes thereby administering a thermal therapy to the prostate; and (d) collecting prostatic fluid in the catheter proximate in time to and/or during the treatment.

Other particular embodiments are directed to methods for treating BPH. The methods include: (a) inserting a catheter with a suction port and associated flow channel and at least one expandable treatment balloon thereon into the urethra of a subject, the treatment balloon positioned to extend outwardly about the perimeter of a portion of the catheter; (b) inflating the at least one treatment balloon, wherein, in position, the inflated treatment balloon takes on a radially expanded configuration and circumferentially contacts targeted tissue in the prostatic urethra; (c) heating a targeted region in the prostatic urethra to a temperature of between about 40-67° C. for a desired treatment time of at least about 20 minutes thereby administering a thermal therapy to the prostate; and (d) collecting prostatic fluid in the catheter proximate in time and/or during the treatment.

The foregoing and other objects and aspects of the present invention are explained in detail in the specification set forth below.

### BRIEF DESCRIPTION OF THE DRAWINGS

FIG. 1 is a schematic section view illustrating a catheter with an expandable treatment balloon in position in the prostatic urethra according to embodiments of the present invention.

FIG. 2 is a block diagram of operations of a method according to embodiments of the present invention.

FIG. 3 is a block diagram of a method of treating the prostate according to embodiments of the present invention.

FIG. 4A is a front view of a catheter according to embodiments of the present invention.

FIG. 4B is a section view of the catheter of FIG. 4A taken about line 4B-4B in FIG. 4A.

FIG. 4C is a section view of an alternate embodiment of the catheter of FIG. 4A similar to the view of FIG. 4B.

FIG. 4D is a section view of yet another alternate embodiment of the catheter of FIG. 4A similar to the view of FIG. 4B.

FIGS. 4E and 4F are schematic illustrations of exemplary bioentry port configurations.

FIG. 5 is a schematic view of the catheter of FIG. 4A operably associated with a suction pump source according to embodiments of the present invention.

FIG. 6A is a front view of a catheter according to alternate embodiments of the present invention.

FIG. 6B is a section view of the catheter taken about line 6B-6B in FIG. 6A.

FIG. 7A is a front view of an additional embodiment of a catheter according to embodiments of the present invention.

FIG. 7B is a front view on another embodiment of a catheter according to embodiments of the present invention.

FIG. 7C is a front view of an alternate configuration of a catheter according to embodiments of the present invention.

FIG. 7D is a section view of the catheter shown in FIG. 7C taken along line A-A according to embodiments of the present invention.

FIG. 8 is a partial cutaway front view of yet another embodiment of a catheter according the present invention.

FIGS. 9A-9D are partial cutaway front views showing another catheter embodiment illustrating the serial progression of different operative configurations according to embodiments of the present invention.

FIG. 10 is a schematic illustration of a system according to embodiments of the present invention.

FIG. 11 is a schematic illustration of a system according to embodiments of the present invention with the catheter in position in the body.

FIG. 12A is a graph of fluid collected over time by a catheter according to embodiments of the present invention.

FIG. 12B is a graph of the volume of fluid collected during a time interval of interest by a catheter according to embodiments of the present invention.

FIG. 13 is a schematic illustration of a set of catheters according to embodiments of the present invention.

### DESCRIPTION OF EMBODIMENTS OF THE INVENTION

The present invention will now be described more fully hereinafter with reference to the accompanying figures, in which preferred embodiments of the invention are shown. This invention may, however, be embodied in many different forms and should not be construed as limited to the embodiments set forth herein. Like numbers refer to like elements throughout. In the figures, certain components, features, or layers may be exaggerated for clarity. In the block diagrams or flow charts, broken lines indicate optional features or steps.

The present invention provides catheters and related systems, methods, and computer program products that can capture biofluids and/or biosamples in vivo, and/or that may enhance the treatment of certain diseases of the body. In other embodiments, the present invention is able to monitor the quality or amount of captured biofluids during or at selected times over the course of a treatment. As examples, a catheter may be used both to collect a sample from a region in the body and to apply a dilatation, internal massage, and/or thermal therapy to a targeted region in a cavity or lumen of the subject. The catheters may be configured with suction capability to enhance the collection of the desired sample or facilitate the efficacy of certain treatments.

The present invention may be particularly suitable for collecting prostatic fluid samples and/or for treating diseases or conditions of the prostate such as BPH, prostatitis, and/or cancer. For prostatitis applications, the present invention may be particularly suitable for treating chronic prostatitis (such as Type II, III or IV, and more particularly the Type III or IV). The present invention may also be suitable for treating prostatodynia. Thus, for ease of discussion, embodiments of the present invention will be primarily discussed in relation to the male urethra. However, other transluminal or transcavity catheter configurations may be used for other lumens or natural body cavities. As such, the catheters of the present invention may be alternately configured and adapted as appropriate for insertion in other natural lumens or body cavities such as, but not limited to, the colon, the uterus, the cervix, the female urethra, the throat, mouth or other respiratory passages, the ear, the nose and the like.

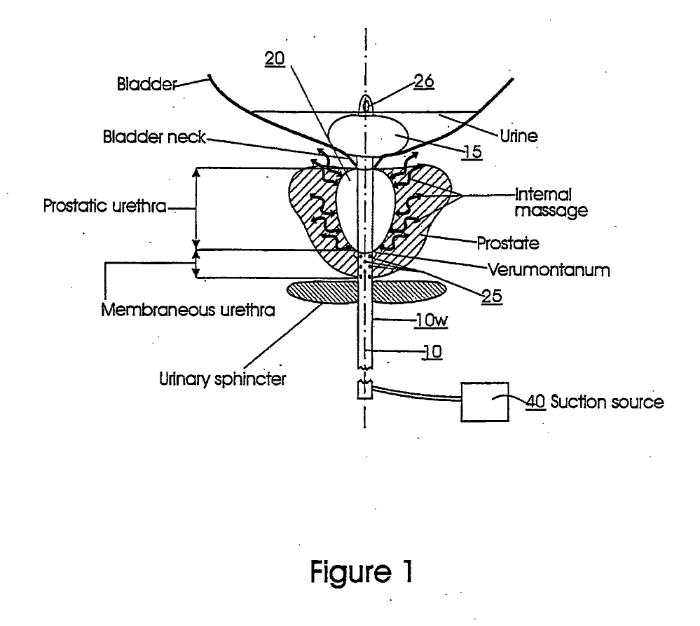

FIG. 1 illustrates a transluminal or transurethral elongated catheter 10 which may be inserted into the prostatic urethra via the penile meatus and up through the male urethra. The catheter 10 can be a flexible catheter so as to be able to be inserted into position in a manner that reduces the likelihood of discomfort (following or bending to the shape of the urethra during insertion). See, e.g., U.S. Pat. Nos. 5,257,977, 5,549,559, and 5,084,044, and U.S. Provisional Patent Application Ser. Nos. 60/248,109, and 60/288,774, the contents of which are hereby incorporated by reference as if recited in full herein.

In the embodiment shown in FIG. 1, the catheter 10 includes a bladder anchoring balloon 15 and at least one treatment balloon 20 positioned on an outer perimeter thereof. The treatment balloon 20 is outwardly expandable. In position, the treatment balloon 20 is configured and sized to radially expand to contact localized tissue in the prostatic urethra. Although shown throughout as a single treatment balloon positioned on a distal portion of the catheter (where a treatment balloon is employed), other configurations can also be employed. For example, the single treatment balloon 20 can be configured as a plurality of circumferentially or axially spaced balloons (not shown). The treatment balloon 20 may be configured as substantially round or oval. The length may be about twice the size of the diameter or width when expanded. The tip of the catheter 10 is shown as linear, but may also have other configurations such as a Coude or Tiemann configuration. The Coude tip may be particularly suitable for some oval treatment balloon configurations (because in the relaxed position the urethra can have an arcuate or oval-like shape (viewed anterior to posterior)).

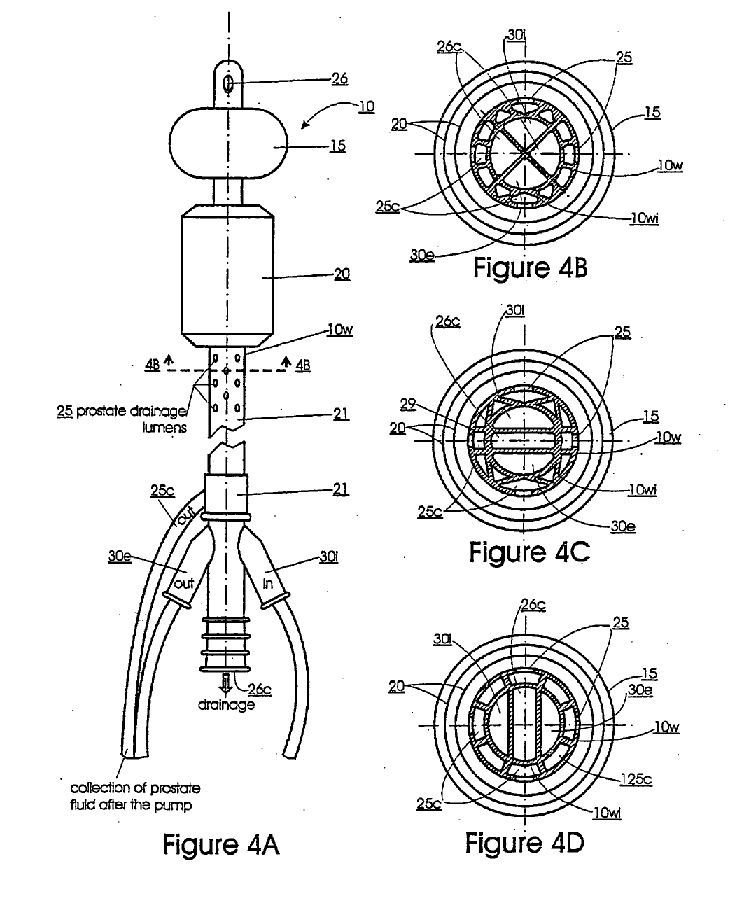

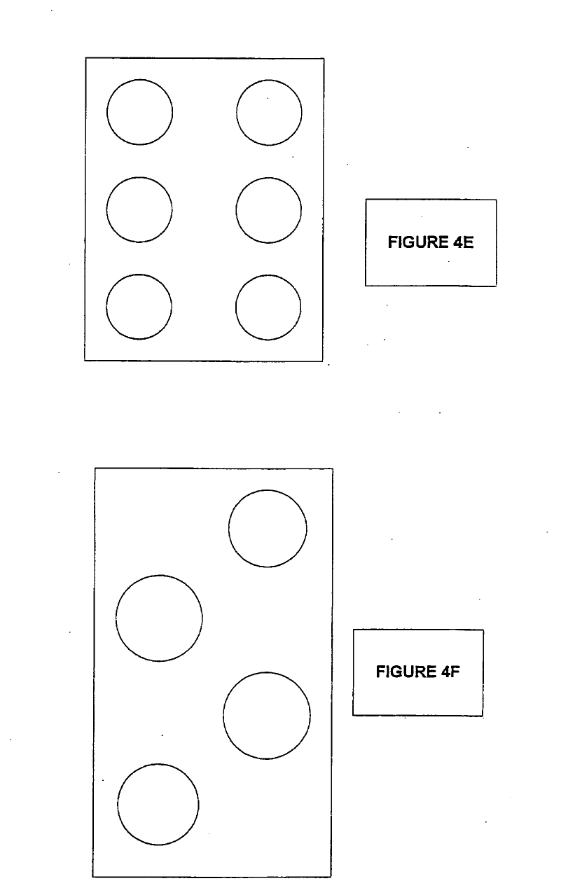

The catheter 10 also includes at least one biosample entry port 25 (shown as a plurality of apertures) formed into the outer wall 10w of the catheter. The biosample entry port(s) 25 is in fluid communication with a biosample flow channel 25c disposed internal to the catheter 10 (FIGS. 4B, 4C). In operation, a suction source 40 can be arranged so as to be in fluid communication with the biosample flow channel 25c to draw in or suction biosamples comprising fluid. The biosample can also include tissue or cell samples. As shown, the biosample comprises prostatic fluid collected from the prostatic urethra and/or the membraneous urethra. As is also shown, in certain embodiments, the biosample entry port 25 may be configured so that, in position in the body, the port or ports 25 reside above the urinary sphincter and below the bladder.

Still referring to FIG. 1, the catheter 10 can include a urine drainage port 26 and associated urine drainage channel 26c. In certain embodiments, the urinary drainage channel 26c can be configured to be separate and in fluid isolation from the biosample flow channel 25c. That is, in position, urine can drain through the catheter 10 via the urine drainage channel 26c while the biosample is suctioned into the catheter biosample flow channel 25c such that each is directed through the catheter without intermingling the two fluids. As such, the collected biosample can be delivered out of the body in a condition so as to be substantially void of urine and in substantially the same concentration with the same constituents as that at its point of entry into the catheter. As used herein, the term “substantially void of urine” means that the collected specimen contains less than about 40% urine. In certain embodiments, the collected specimen contains less than about 10-20% urine. In yet other embodiments, the specimen is collected in a manner such that it is not diluted from its in vivo concentration by more than 1-5%.

In other embodiments, the bladder-anchoring balloon 15 can be configured to substantially block or plug the urethra at the bladder neck so as to inhibit the entry of urine into the prostatic urethra during collection of the prostatic biosample (not shown).

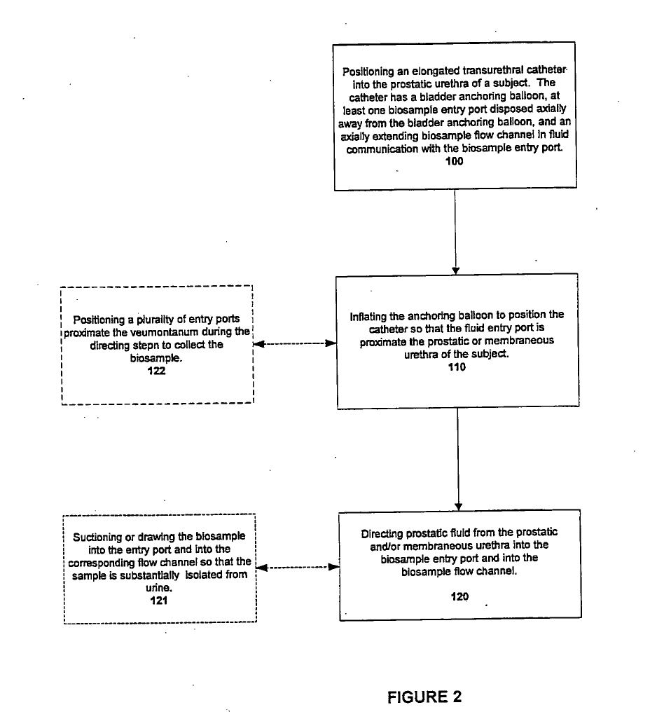

FIG. 2 illustrates operations that can be performed according to embodiments of the present invention. An elongated transurethral catheter can be positioned into the prostatic urethra of a subject. The catheter can include a bladder anchoring balloon, at least one biosample entry port disposed axially away from the bladder anchoring balloon, and an axially extending biosample flow channel in fluid communication with the biosample entry port (block 100). The anchoring balloon can be inflated to position the catheter so that the fluid entry port is proximate the prostatic or membraneous urethra of the subject (block 110). A biosample comprising prostatic fluid can be directed from the prostatic and/or membraneous urethra and/or into the biosample entry port and into the biosample flow channel (block 120). The directing step can be carried out by suctioning or drawing the biosample into the catheter (block 121). In addition, the at least one biosample entry port can be a plurality of entry ports that are positioned to reside proximate the verumontanum during the collection of the biosample (block 122).

Referring again to FIG. 1, in certain embodiments, the catheter 10 can be configured to deliver a thermal therapy in addition to being configured to suction a biosample. In certain embodiments, the thermal therapy can be applied to targeted tissue at a temperature of between about 40-85° C. (or higher for some applications), and is typically between about 40-67° C. The therapy time can be carried out in desired time increments according to the particular application, typically ranging between about 5 minutes to 90 minutes. The administration of a thermal therapy can enhance the quantity of a biosample specimen that can be collected. In certain embodiments, suctioning fluid biosamples during the thermal therapy may enhance the efficacy of the treatment.

The heat can be supplied by any desired heating source including RF, microwave, laser, ultrasound, conductive heat that can be generated with localized or circulating heated fluid, and the like. For example, the heat can be applied using microwave and RF energy to heat the tissue (which may include a distal heating element) and expanding the treatment balloon a desired distance as it resides in the prostatic urethra to provide the internal thermal therapy.

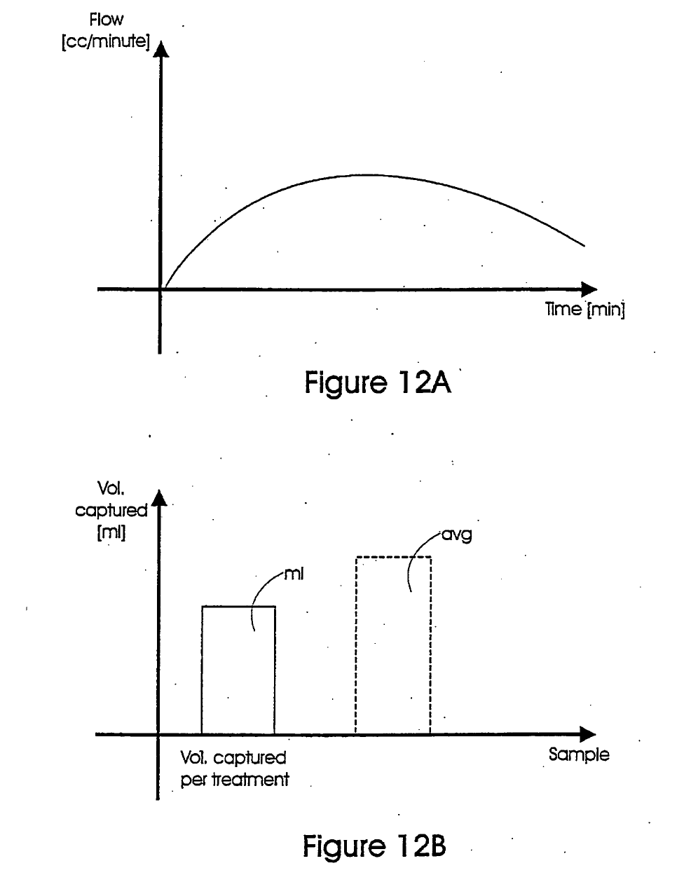

The collection of the biosample can be carried out in a number of ways, such as concurrently with the administration of a thermal therapy. The collector can also be carried out intermittently during the course of the treatment as well as either before and/or after the treatment. In other embodiments, the collection can be performed substantially continuously for a major portion or all of the treatment. FIG. 12A illustrates that the flow rate or volume of collected biosample over time can be monitored over the treatment period. This data may indicate when to end the therapy, or what the tissue activity is in the body in the treatment region. FIG. 12B illustrates that the biofluid can be collected for analysis. It may be possible to develop a predictive behavior, for example, if a lesser volume is obtained relative to standardized norms (set by the subject or a correlated population), this may indicate that the penetration depth of the treatment is reduced or that the tissue is deficient in liquid.

Of course, with or without the use of a thermal therapy, the collected biosample can be analyzed for the presence, absence or elevated or deficient levels of one or more analytes of interest to assess the therapeutic response of the subject to a treatment such as a medicament (whether thermal, pharmaceutical, diet, exercise or other behavioral based regimen) or to provide a diagnosis of a condition. Thus, the catheter can be alternatively configured to provide the desired access to the desired tissue. The catheter can also be configured to administer a desired medicament whether pharmaceutical or sterile liquid and the like. The medicaments can include, but are not limited to, one or a mixture of antibiotics, anti-inflammatory medication, antioxidants (such as QUERCETIN), anesthetics or pain relief medications, and sterile water.

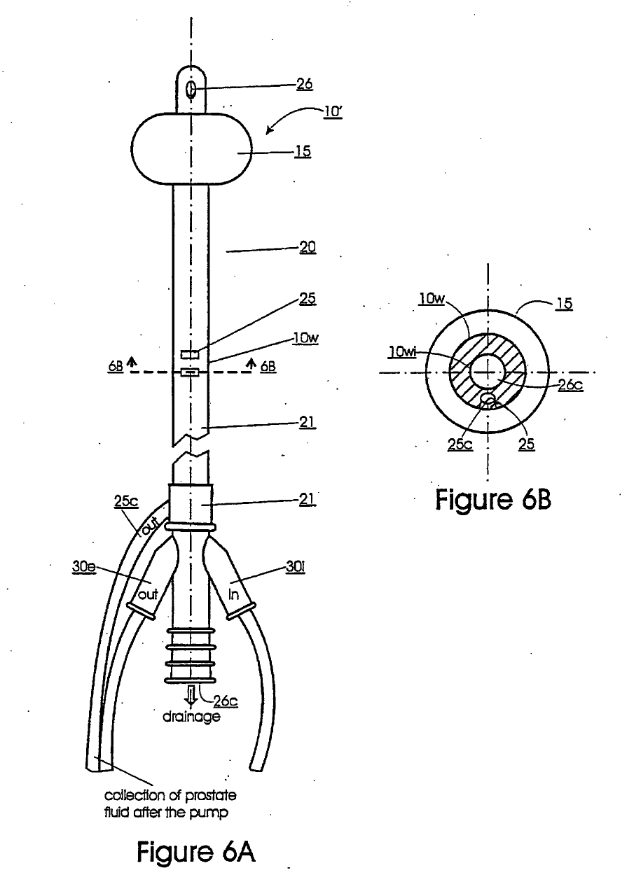

For example, as shown in FIG. 6A, the catheter 10′ does not include a treatment balloon and obtains the biosample without the use of a concurrent internally administered thermal therapy or an internal massage. Of course, this embodiment does not preclude the use of external or rectal massages and the like that can be performed during and/or proximate in time to the capturing in vivo of the flowably collected biosample.

In certain embodiments, the heat or thermal therapy is supplied by heating fluid external of the body of the subject and directing it so that it travels captured in the catheter to the treatment balloon. In these embodiments, the system and the balloon can be configured to continuously circulate heated fluid to a regulated desired thermal treatment temperature. As such, the catheter can have increased insulation regions located about the shaft below the treatment balloon to insulate the non-targeted tissue as the heated fluid travels to the remote in vivo treatment site.

In particular embodiments, the catheter 10 can be configured to deliver a thermal ablation treatment to a targeted region (shown by the arrows in the lined region in the prostate in FIG. 1). The term “thermal ablation” refers to exposing the targeted tissue to a temperature that is sufficient to kill the tissue. In certain embodiments, the thermal ablation is carried out by exposing the targeted tissue to thermocoagulation via circulating hot liquid heated external of the body of the subject and directed to expand the treatment balloon 20 in the targeted treatment region. For thermal ablation therapies, the tissue is exposed to an elevated temperature that is greater than (or equal to) about 45° C. for a predetermined period of time such as about 5-20 minutes or longer.

In certain embodiments, the thermal ablation is directed to treating BPH and the thermal therapy is carried out so that the prostatic tissue is exposed to a temperature of about 60-62° C. for a treatment period that is about 20-90 minutes in duration, and more preferably about 45 minutes. In other embodiments, the treatment is directed at prostatitis and the targeted tissue is exposed to elevated temperatures in the range of about 40-47° C. (at or below minimal ablation temperatures) for a period of about 20-90 minutes. The prostatitis thermal treatment and/or the BPH thermal ablation therapy can be carried out in a localized treatment region within the prostatic urethra, the treatment region being generally described as including the prostatic urethra below the bladder neck and above the verumontanum of the subject. Alternatively, the treatment region may include the bladder neck or a portion of the bladder neck itself.

An example of a thermal treatment system that is configured to circulate heated fluid to administer water induced thermotherapy is identified as the Thermoflex® system available from ArgoMed, Inc. located in Cary, N.C. See also, U.S. Pat. Nos. 5,257,977 and 5,549,559 to Eshel, and co-assigned U.S. patent application Ser. No. 09/433,952 to Eshel et al, the contents of which are hereby incorporated by reference as if recited in full herein.

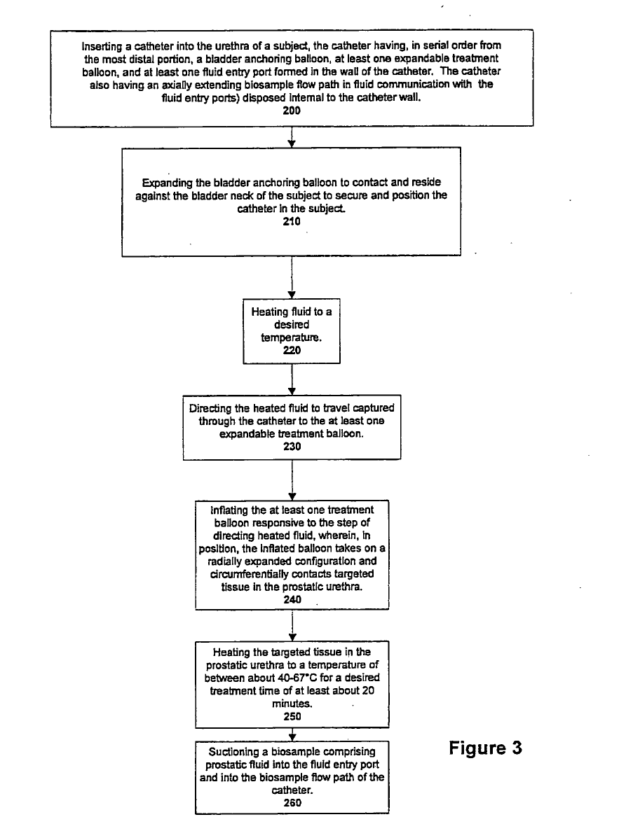

FIG. 3 illustrates a flow chart of operations according to one embodiment of the present invention. As before, a catheter can be inserted into the urethra of a subject. The catheter can include, in serial order from the most distal portion, a bladder anchoring balloon, at least one expandable treatment balloon, and at least one fluid entry port formed into the wall of the catheter. The catheter also includes an axially extending biosample flow path in fluid communication with the fluid entry port(s) disposed internal to the catheter wall (block 200). The bladder-anchoring balloon can be expanded to contact and reside against the bladder neck of the subject to secure and position the catheter in the subject (block 210). Fluid can be heated to a desired temperature (block 220) and directed to travel, captured, through the catheter to the at least one expandable treatment balloon (block 230). The at least one treatment balloon inflates responsive to the step of directing heated fluid. In position, the inflated balloon takes on a radially expanded configuration and circumferentially contacts targeted tissue in the prostatic urethra (block 240). The targeted tissue in the prostatic urethra can be heated to a temperature of between about 40-67° C. for a desired treatment time of at least about 20 minutes (block 250). A biosample comprising prostatic fluid can be suctioned into the fluid entry port and into the biosample flow path of the catheter (block 260). The sample can be analyzed as noted above.

Referring to FIG. 4A, the catheter 10 includes an outer wall 10w, the anchoring balloon 15, the treatment balloon 20, and an elongated shaft 21. The catheter 10 also includes inlet and outlet or exit fluid circulating paths 30i, 30e, respectively, as well as a urinary drainage channel 26c (which can also be used to deliver medicaments therethrough while the catheter 10 is in position in the subject by reversing the direction of flow through the channel). The catheter 10 can also include a collar 10c on its proximal end with four separate fluid flow paths 26c, 25c, 30e, 30i. The collar 10c connects the passageways/lumens or channels of the distal portion of the catheter into the desired flow paths external of the body (shown as comprising flexible conduits on the other side of the collar 10c).

The anchoring balloon 15 can be in fluid communication with the treatment balloon 20 such that both are inflatable by the circulating heated fluid. Alternatively, the balloons 15, 20 can be in fluid isolation (and separately inflatable). The upper anchoring balloon 15 can be separately inflatable to allow this balloon 15 to be inflated before the treatment balloon 20. This can reduce the likelihood that the balloon 15 or 20 will be inflated below the desired location (potentially introducing damage to the bladder neck or the upper portion of the prostate urethra) and facilitate proper positioning of the catheter 10 in the prostate relative to the bladder.

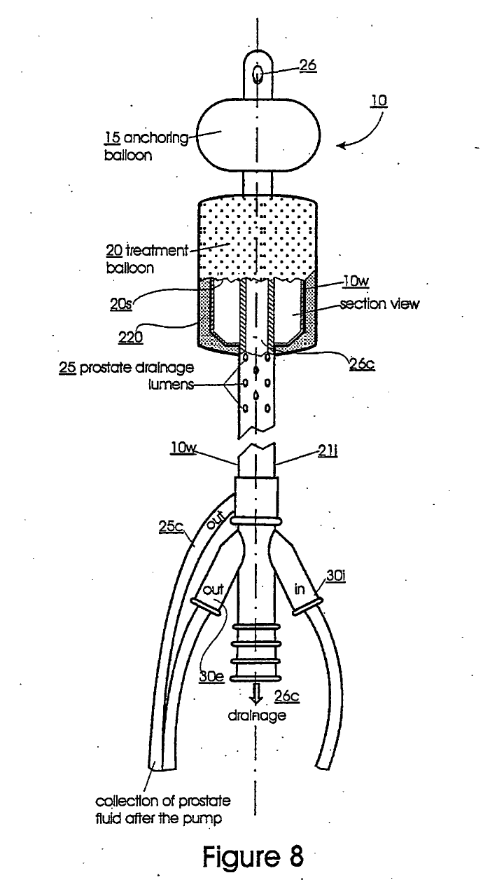

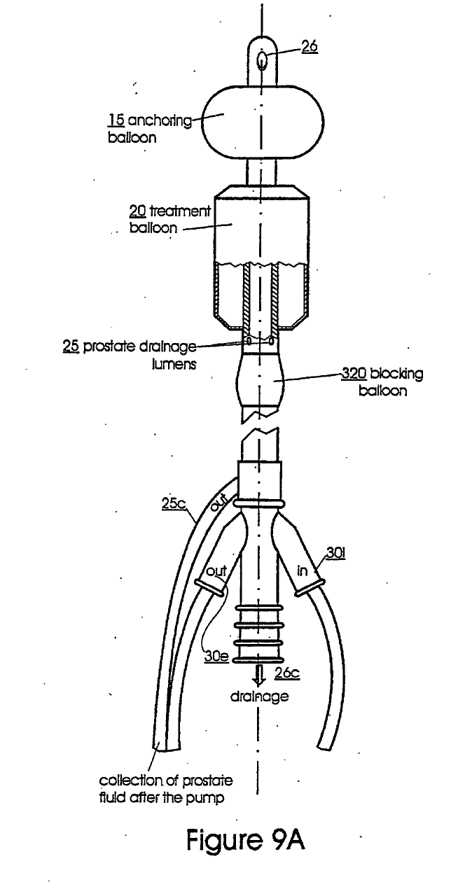

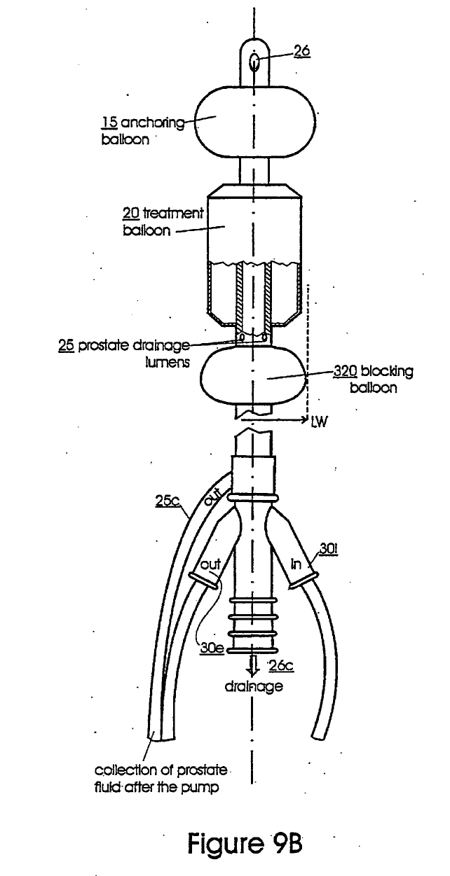

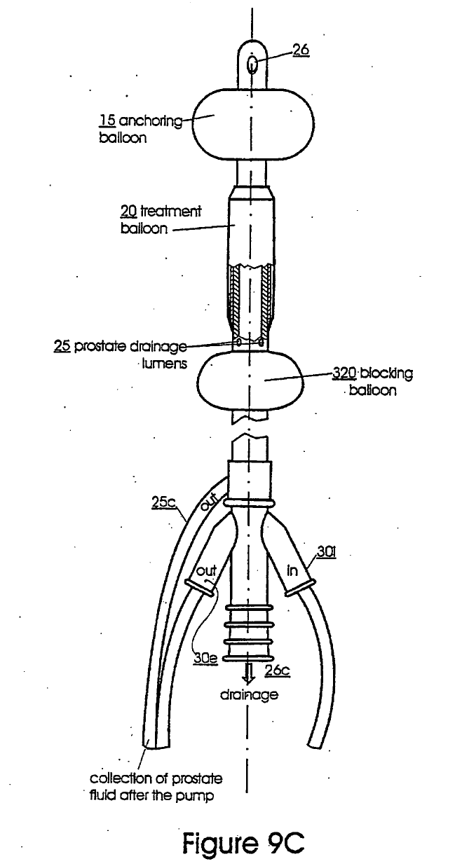

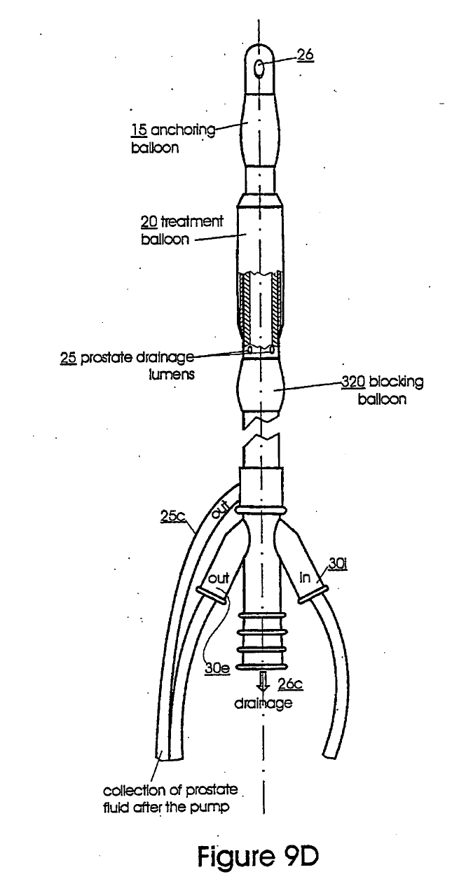

Turning to FIG. 4B, heated fluid is heated external of the subject, directed into the catheter 10 and circulated in the enclosed fluid paths 30i, 30e in the catheter 10. The fluid is directed through the shaft 21 via the inlet path 30i to the treatment balloon 20 located proximate the desired treatment site, then out of the treatment balloon 20 to the outlet path 30e and out of the subject. The system can be configured to operate with a low volume of liquid. The term “low volume” means below about 100 ml, and, in conventional circulating systems can be in the range of about 20-50 ml. In certain particular embodiments, about 20-40 ml of liquid can be circulated at any one time in the catheter. The system itself may be configured to hold an additional quantity, such as about 35 ml or more, in reserve. Over time, additional quantities of liquid can be introduced into the circulating fluid loop. In any event, typically, about 20-100 ml of liquid can be contained in the closed loop system, a portion or all of which can be circulated and heated during operation. An example of a closed loop system 50 is shown in FIGS. 8 and 9, the closed loop system being able to deliver a thermal treatment via the treatment catheter 10.

The circulating fluid (and the anchoring balloon inflation media, when separately inflatable) is preferably selected to be non-toxic and to reduce any potential noxious effect to the subject should the balloon integrity be compromised, accidentally rupture, leak, or otherwise become impaired during service.

The catheter 10 is preferably flexibly configured so as to be able to bend and flex to follow the shape of the lumen (even those with curvatures) as it is introduced into the lumen until a distal portion of the catheter 10 reaches the desired treatment site. It is also preferred that the catheter 10 is configured such that it can flex to follow the contours of the male urethra while having sufficient rigidity to maintain a sufficiently sized opening in the drainage (preferably the central) lumen 26c to allow urine drainage and or flushing or drug delivery during the initial healing period while in position (even after exposure to the thermal ablation therapy described above).

The catheter 10 can be sized with a relatively small cross-sectional area with a thin outer wall 10w so as to be able to be inserted into and extend along a length of the desired lumen to reach the desired treatment site. As used herein, the term “thin outer wall” means a wall having a thickness of about 3 mm or less, and preferably about 2 mm or less. For prostate applications, the cross-sectional width of the catheter 10 is typically less than about 100 mm and, more typically, the width or outer diameter of the catheter 10 is about 6-9 mm.

Referring to FIG. 4B, a major portion of the cross-sectional area of the shaft region 21 of the catheter 20 is taken up by the size of the fluid channel(s) held therein.

The catheter 10 can include only a single internal fluid channel, such as the biosample flow channel 25c or the biosample flow channel 25c, and one or more additional channels. As shown in FIG. 4B, for certain applications, the catheter 10 includes at least four separate fluid channels: the biosample flow channel 25c; the circulating inlet and outlet channels 30i, 30e; and the fluid drainage channel 26c. As shown in FIG. 4B, the urine drainage channel 26c may be disposed intermediate the circulating inlet and outlet channels 30i, 30e. As is also shown, the biosample entry ports 25 can be circumferentially spaced apart about the outer wall 10w of the catheter and terminate into a common biosample flow channel 25c.

Alternatively, the catheter 10 can be configured to include a plurality of separate biosample flow channels 25c, each corresponding to one or more entry ports 25. For example, FIG. 4D illustrates that the catheter 10 includes a plurality of elongated channels 125c that are positioned between the inner lumens and the outer wall 10w. The elongated channels 125c can be configured as a plurality of separate axially extending elongated tubular members with relatively small internal diameters that circumferentially span the internal lumen passage(s). One or more of the tubular members can act as the biosample flow channel 25c. As shown by the shaded channels, this example uses three biosample flow channels 25c. Other numbers and locations of the biosample flow channels can also be used. In addition, each biosample flow channel can be in fluid communication with one or a plurality of entry ports 25. The entry ports 25 can be formed to be axially and/or laterally spaced apart about a selected perimeter portion of the catheter. FIG. 4E illustrates that the entry ports can be axially and laterally aligned about the perimeter of the catheter 10 while FIG. 4F illustrates that the entry ports 25 can be configured to be offset one from the other. Other configurations can also be employed.

In particular embodiments, as shown in FIG. 4B, a common biosample flow channel 25c is configured to encase the urine drainage channel 26c as well as the circulating inlet and outlet channels 30i, 30e.

FIG. 4C illustrates a different fluid lumen and wall configuration. As shown, the biosample entry ports 25 are arranged to lie within a baffle structure 29 that radially extends from an inner wall 10wᵢ to the outer wall 10w to provide lateral structural reinforcement that can provide resistance to closure during operation. As is also shown, the baffle structure 29 is configured in a “V” or laterally extending triangulated or pointed structure. Other baffle or support configurations can be used to laterally reinforce or bolster an open biosample flow channel 25c during operation. For example, a thicker outer and/or wall 10w, 10wᵢ can be used about the region of the biosample ports 25 of the catheter. In addition, the apertures or ports 25 may also be formed outside the baffle structure 29 (i.e., outside the “V”). In addition, as shown, the inner fluid lumens (the drainage channel 26c, and the inlet and outlet channels 30i, 30e) can be alternately arranged and configured. As shown the inner fluid channels 26c, 30i, 30e, are three substantially equal pie-shaped wedges. These are merely examples of wall and lumen configurations and other configurations may also be used as contemplated by the present invention.

FIGS. 6A and 6B illustrate a catheter 10′ with at least one slotted port 25 that is in fluid communication with the internal discrete biosample flow channel 25c. As shown, the catheter 10′ can also include the urinary drainage channel 26c. The discrete biosample flow channel 25c may be defined by an insert or tube positioned in the catheter 10 and formed of a material having increased rigidity over the wall of the catheter 10w. For example, a PVC (polyvinylchloride) insert can be disposed between the inner and outer wall 10w, 10wᵢ. Additional discrete or connected channels 25c can also be employed.

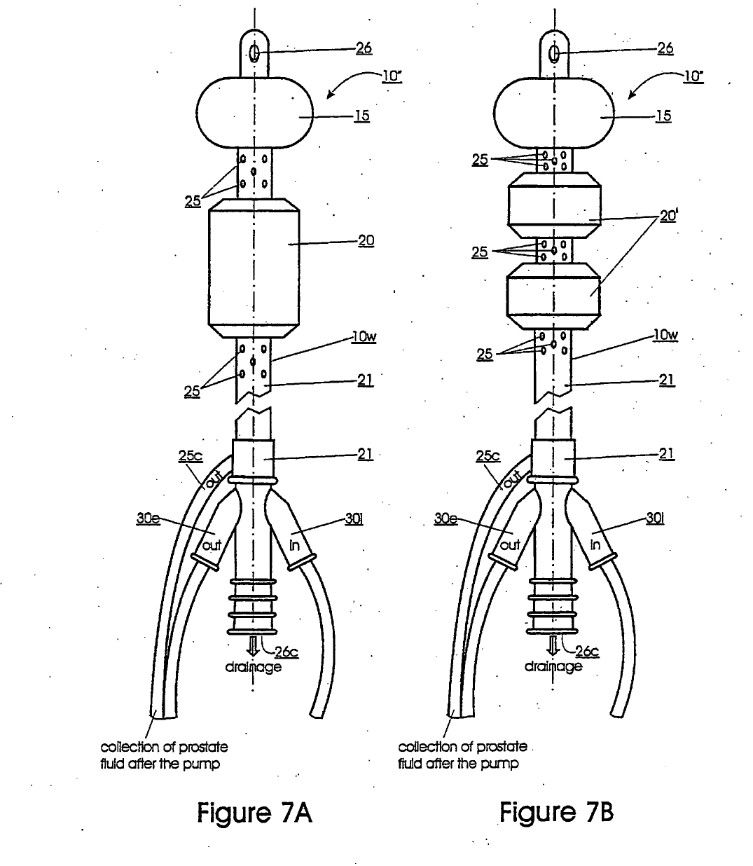

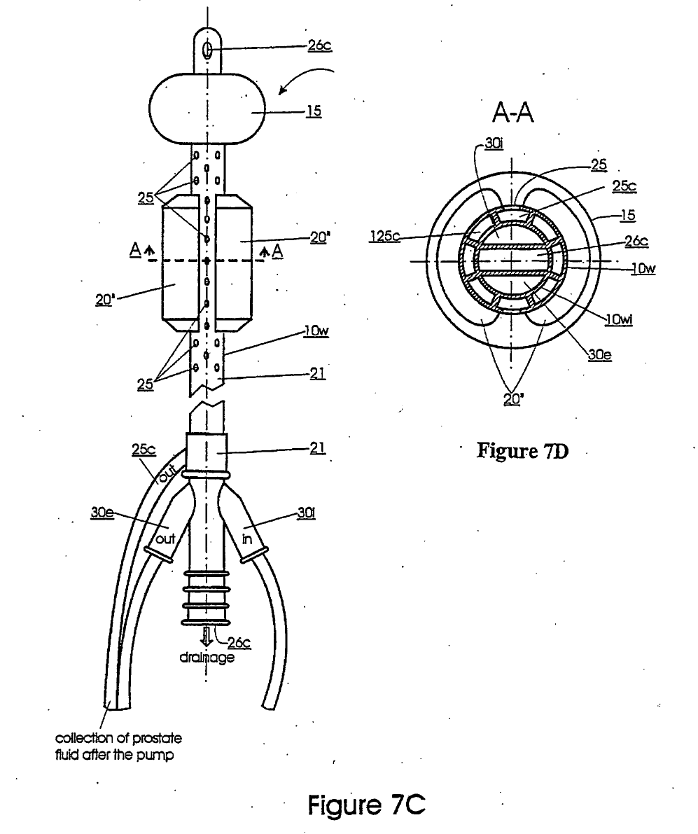

FIG. 7A illustrates the catheter with biosample collection ports 25 disposed above and below the treatment balloon 20 on the catheter 10″. FIG. 7B illustrates that the treatment balloon 20′ may be configured to extend about portions of the perimeter of the catheter shaft so as to be axially intermittently spaced expandable balloons 20′ with biosample ports 25 located at one or more of above, below, and intermediate thereof. FIGS. 7C and 7D illustrate that the treatment balloon 20″ can be configured as radially spaced apart expandable balloon segments 20″ and the biosample ports 25 can be located radially spaced apart between the balloon segments 20″. As such, the biosample ports 25 may be positioned so as to be axially or laterally interspersed or intermediate the expandable treatment balloons 20′, 20″.

The catheter 10 may also include a region with increased insulation 21i (FIGS. 8, 11) encasing fluid channels 30ᵢ, 30e, 26c. The increased insulation region 21i can reduce the temperature that non-targeted tissue is exposed to along the fluid flow paths in the catheter 10. The increased insulation regions 21i can extend along the portion or length of the catheter that, in operation, resides below or away from the targeted treatment region in the body (below the sphincter in the male urethra in the prostate application as shown in FIG. 1) during the thermal therapy to reduce the likelihood that the non-targeted tissue will be exposed to undue elevated temperatures.

The increased insulated regions 21i have been provided by various means such as configuring the catheter with an extra layer or thickness of a material along the proximal or lower shaft portion. Other insulation means include a series of circumferentially arranged elongated channels or conduits (either filled with air or other material (and that may be sealed enclosures of same), or which are configured to provide lateral thermal resistance), which encircle the heated circulating fluid passages and provide thermal insulation along the elongated shaft portion of the catheter. Additional description and examples of insulation means and configurations, wall structure configurations, and lumen/channel configurations that may be collapse-resistant during operation are found in U.S. Pat. Nos. 5,257,977 and 5,549,559 to Eshel, and co-pending and co-assigned U.S. Provisional Patent Application Ser. No. 60/248,109, the contents of which are hereby incorporated by reference as if recited in full herein.

In any event, as the heated fluid travels through the fluid circulating passages, the insulation means acts to reduce the heat transferred to non-targeted treatment sites, such as along the penile meatus, urethral mucosa, or urethral sphincter, during the treatment (such as BPH, prostatitis, or cancer therapies).

In certain embodiments, the catheter 10, 10′, 10″ can have an outer wall 10w and an inner wall 10wᵢ, each having a thickness of between about 1-2 mm formed of a thermoplastic elastomer such as silicone, rubber, plasticized PVC, or other suitable biomedically acceptable elastomeric body.

FIG. 8 illustrates another embodiment of the present invention. As shown, the catheter 10 can include a sleeve 20s disposed over the treatment balloon 20. The sleeve may also extend to cover the anchoring balloon (not shown). The sleeve 20s can compress the treatment balloon 20 to take on a low profile (held snugly against the shaft) during insertion and removal of the catheter from the body. As such, the sleeve 20s may be configured from flexible thin material that is able to compress the treatment balloon 20 against the shaft of the catheter. The sleeve 20s can be configured so that it is able retain its elasticity after exposure to a thermal treatment so that it can cause the treatment balloon to collapse against the shaft when the circulating fluid is removed in preparation for sliding removal from the body. In certain embodiments a fluid or media 220 can be disposed intermediate the treatment balloon 20 and the sleeve 20s (shown as the shaded area intermediate same in the figure). This fluid or media 220 can be a medicament that leaches into the body over the course of the treatment.

As shown by the dotted shading on the sleeve 20s, the sleeve can be configured as a porous material (or with drug exit ports) that can allow the internally held media or fluid 220 to exit the sleeve 20s during or after treatment. That is, instead of pre-filling the catheter with the medicament or media 220 during fabrication, the medicament or media 220 can be directed into the catheter and then into the sleeve 20s (via a corresponding flow path) during or after administration of the thermal therapy and then released into the body of the subject through the porous membrane.

Alternatively, a selected coating can be disposed over the treatment balloon or sleeve such that it can be administered or released in vivo during the treatment. For additional descriptions of suitable sleeves, materials, and media, see co-pending and co-assigned U.S. Provisional Patent Application Ser. No. 60/288,774, the contents of which are hereby incorporated by reference as if recited in full herein.

In other embodiments, the prostate drainage port(s) 25 and channel(s) 25c can be used to administer medicaments sterile liquids and the like at desired times before, during, or after the treatment. For example, one or more of the elongated channels 125c shown in FIG. 4D can be used to direct flowable medicines into the prostatic urethra. The same or different ones of the elongated channels 125c can be used to aspirate and/or deliver medicaments. The elongated channels 125c can also define or form a part of the insulation means for the increased insulation regions 21i on the catheter. Other medication ports and channels can be formed into the catheter as desired.

In certain embodiments, the catheter 10 can be configured to provide an occlusion or segmented region in the urethra in which to administer a medicament. This segmentation can direct the medicament into the desired region and effectively trap the medicament there so as to inhibit its run-off or exit from the treatment region. In particular embodiments, the entry ports 25 and associated channels 25c are used to introduce a desired medicament at elevated pressures to the prostate proximate in time to the dilatation and heat treatment. As such, the catheter 10 can include a blocking balloon 320 (FIGS. 9A-9D) that is configured to close off the lower portion of the urethra from drainage except for the urinary flow through the catheter. See e.g., U.S. Pat. No. 5,419,763, the contents of which are hereby incorporated by reference as if recited in full herein. The blocking balloon can be configured so that in position it expands to contact the membraneous urethra (up to the sphincter) or the bulbous urethra (down to the sphincter).

FIGS. 9A-9D illustrate a sequence of operations for a catheter having an example of a blocking balloon 320. As shown, the blocking balloon 320 is positioned below the prostate drainage ports 25. In particular embodiments, the blocking balloon 320 is positioned on the catheter 10 such that it is below the anchoring balloon 15 (that engages with the bladder to hold the catheter in position in the body) and so as to reside at a desired location (typically proximate to but above or below the urinary sphincter) when properly positioned in the subject.

FIG. 9A illustrates the configuration of the treatment balloon 20 and the blocking balloon 320 during administration of a thermal therapy (and the anchoring balloon 15 also expanded to hold the catheter 10 in its desired position in the body). In other embodiments, the blocking balloon 320 can also be expanded during the entire or selected portions of the therapy. FIG. 9B illustrates the blocking balloon 320 having an expanded configuration. As shown, the blocking balloon 320 can have a lateral expansion width L_{w} that is about equal to or larger than the treatment balloon 20. FIG. 9C illustrates that the treatment balloon 20 can be collapsed before the blocking balloon 320. In certain embodiments, a medicament is directed to flow in the channel 25c and exit the ports 25 at an elevated pressure so that it may have increased penetration depth into the prostate proximate in time to the dilatation and heat treatment. The elevated pressure may be selected so that the medicament is expelled from the port 25 with sufficient force to promote tissue penetration and spraying of proximate tissue. The expulsion pressures may be between about 0.1-7 atm, and can, in particular embodiments, be from about 1-2 atm.

FIG. 9D illustrates the configuration of the catheter upon insertion or removal with the anchoring balloon 15, the treatment balloon 20, and the blocking balloon 320 all collapsed against the shaft of the catheter.

In certain embodiments, the therapeutic treatment delivered by the thermal system can include an internal massage that is delivered by repetitively outwardly expanding and then contracting the treatment balloon 20 a desired distance. See co-pending and co-assigned U.S. Provisional Application Ser. No. 60/308,344, the contents of which are hereby incorporated by reference as if recited in full herein. In certain embodiments, the system can be configured to provide a relative quick massage cycle (such as about 1-12 cycles or pulses every second) or slower massage cycle (20-60 pulses per minute); the rate and force of the massage can be adjusted during the treatment as will be discussed further below.

In addition, fluid or air provided at a non-elevated (ambient or body) temperature may be used to perform the massage (or an initial portion of the massage) with or without a thermal therapy to relax the local tissue prior to, after, or concurrent with suctioning the biosample from the targeted biosample region.

The terms “medicament” and “therapeutic agent” include medicines, food supplements, or bioactive substances or formulations used to treat diseases or symptoms. For diseases or conditions of the prostate such as cancer, BPH, and prostatitis or symptoms associated therewith, the therapeutic agent can be delivered either systemically or locally alone or as an adjunct to the thermal or massage therapy, including over-the-counter or prescription pharmaceutical products, vitamins or food, beta radiation, and the like.

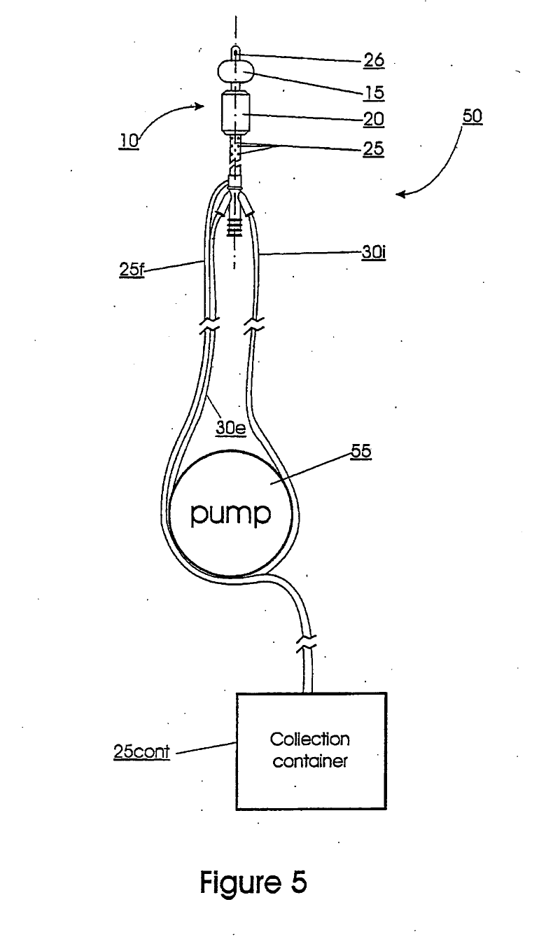

FIG. 5 illustrates that the thermal treatment system 50 can be configured as a closed loop circulating fluid system. In addition, this embodiment shows that the suction source 40 can also be the fluid circulation pump 55. The biosample flow path 25c can include a length of flexible conduit 25f that extends from the catheter external of the body and engages with the pump mechanism 55. The pump 55 draws or directs the fluid therein to discharge downstream of the pump 55 into a biosample collection chamber or container 25cont to provide the suction force in the biosample flow path 25c. See e.g., U.S. patent application Ser. No. 09/433,952 and U.S. Pat. No. 5,549,559, the contents of which are hereby incorporated by reference as if recited in full herein, for descriptions of a suitable closed loop circulating fluid system. Fluid circulating WIT™ catheters with expandable treatment balloons are available from ArgoMed, Inc., in Cary, N.C. The pressure in the treatment balloon (which corresponds to the pressure in the closed loop system) may be from about 0.5-4 atm, and typically at least about 0.75-2 atm during at least a portion of the treatment to increase the pulsation force presented to the localized tissue.

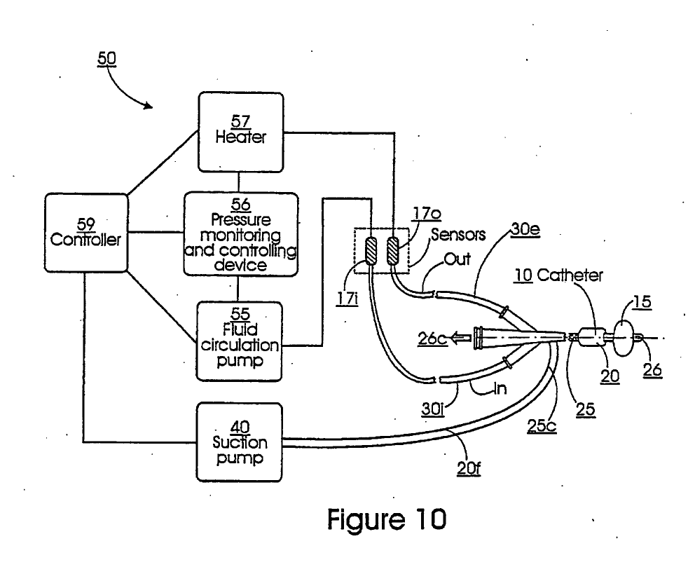

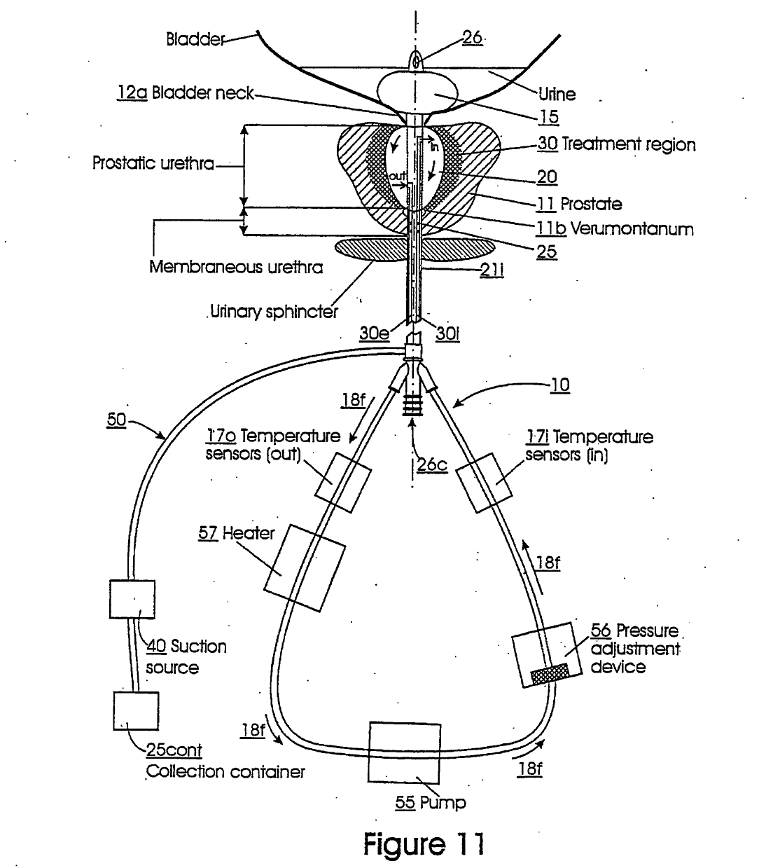

FIG. 10 illustrates one embodiment of a system 50 which can be used to collect and/or suction prostatic fluid from biosample ports 25 and that may, in certain embodiments, also heat fluid that is then directed into the catheter 10 to cause the treatment balloon to expand so as to apply a thermal and/or massage therapy to the localized tissue in the prostatic urethra. As shown, the system 50 is a closed loop system includes a fluid circulation pump 55, a pressure monitoring and controlling device 56, a heater 57, a controller 59, and temperature sensors 17i, 17o, all of which are operably associated with the catheter 10. As noted above, the system can be configured as a low volume system (circulating from between about 10-100 ml of fluid). A suitable closed loop system known as the Thermoflex® System is available from ArgoMed, Inc. in Cary, N.C.

In certain embodiments, the circulation and/or the internal massage can be provided by using a peristaltic pump to generate pulsatile fluid flow. A three-roller pump may be configured to operate to provide about 1-12 or 1-20 expansion and contraction pulses per minute in the balloon. This action can be caused by using a pulsatile flow pump having three rollers with between about 200-750 rotations per minute while a two roller pump may be configured to operate with between about 200-500 rotations per minute, each can operate so as to provide a corresponding number of pulses to the treatment balloon. Suitable pump heads are available from Watson Marlow Inc., of Wilmington, Mass., and Barnant Co., of Barrington, Ill. Of course, other methods for expanding and contracting a treatment balloon or generating the pulsatile flow can also be used as will be appreciated by those of skill in the art. As shown in FIG. 5, the biosample flexible conduit 25f may be configured to wrap about the pump head/rollers to provide the suction source or other suction sources such as a stand-alone suction pump or piston may also be used.

FIG. 11 illustrates the catheter 10 in position in the body and operably associated with an associated operating system 50 according to one embodiment of the present invention similar to that shown in FIG. 10. In this embodiment, the catheter 10 is configured to collect the biosample, but may not employ a suction source. Rather, gravity, capillary action, or other collecting means may be employed. It is also shown that the entry ports 25 are positioned to reside proximate the verumontanum of the subject where increased prostatic fluids may be more prevalent or easier to collect. Of course, the entry ports 25 can be alternatively configured on the catheter 10 depending on the targeted region of interest.

In FIG. 11, the circulating fluid flow in the system and catheter is shown by the arrows 18f. The pressure adjustment device 56 may also include a pressure sensor 15s to sense pressure in the system and be configured for automatic pressure adjustment to facilitate consistency between treatments and may also be configured to allow the patient to set certain operating pressures in the balloon 20 and/or system 50. Additional description of pressure adjustment systems and devices can be found in co-pending, co-assigned U.S. Provisional Application Ser. No. 60/318,556, the contents of which are hereby incorporated by reference as if recited in full herein.

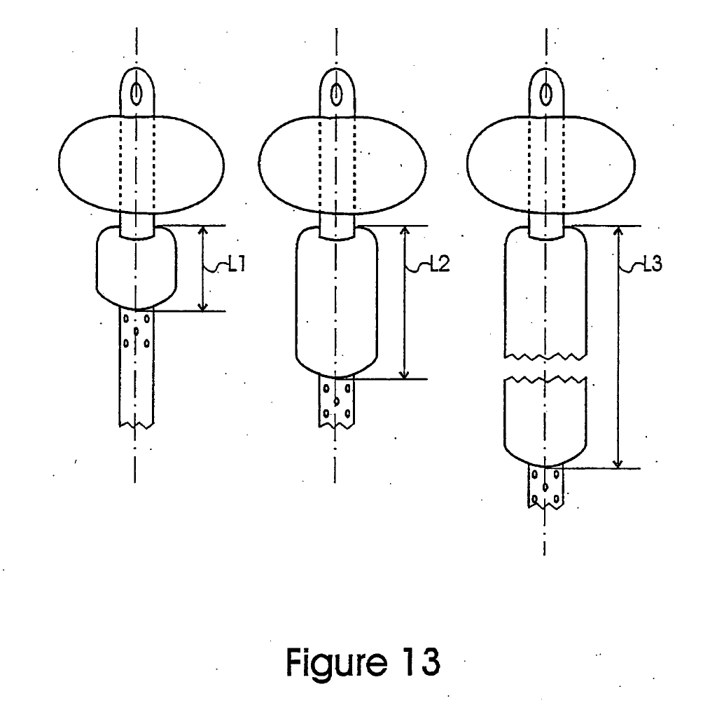

As shown in FIG. 1, in certain embodiments, the treatment balloon 10 is configured with an axial length that is selected so that, in position, it resides above the verumontanum of the subject. Thus, the catheters 10, 10′, 10″ may be provided as a kit or set of catheters as shown in FIG. 13 with various lengths of treatment balloons 20 with the suction ports 25 positioned thereon. The catheters may be configured in an array of different treatment balloon 20 sizes and/or lengths to provide a custom fit for the subject (the length of the prostatic urethra will vary subject to subject). In particular embodiments, a set of catheters can be provided such so as to provide, in about 0.5 cm increments, treatment balloon lengths ranging from about 1 cm to 6 cm such that, in operation, a selected treatment balloon resides above the verumontanum of the subject in the prostatic urethra.

The concurrent combination of suction with or proximate in time to the administration of one or more of pressure (such as massage therapy or balloon dilatation) and/or heat may provide increased therapeutic responsiveness over conventional treatments or may provide improved sample collection techniques. In other embodiments, as an alternative to thermal treatment, the internal massage can be administered alone by using a low heat or cooled or ambient non-heated medium such as water or other biocompatible substance to cause the treatment balloon to expand and suctioning the prostatic urethra or membraneous urethra.

As will be appreciated by one of skill in the art, the present invention may be embodied as a method, data or signal processing system, or computer program product. Accordingly, the present invention may take the form of an entirely hardware embodiment, an entirely software embodiment or an embodiment combining software and hardware aspects. Furthermore, the present invention may take the form of a computer program product on a computer-usable storage medium having computer-usable program code means embodied in the medium. Any suitable computer readable medium may be utilized including hard disks, CD-ROMs, optical storage devices, or magnetic storage devices.

The computer-usable or computer-readable medium may be, for example but not limited to, an electronic, magnetic, optical, electromagnetic, infrared, or semiconductor system, apparatus, device, or propagation medium. More specific examples (a nonexhaustive list) of the computer-readable medium include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted or otherwise processed in a suitable manner if necessary, and then stored in a computer memory.

Computer program code for carrying out operations of the present invention may be written in an object oriented programming language such as Java®, Smalltalk, Python, or C++. However, the computer program code for carrying out operations of the present invention may also be written in conventional procedural programming languages, such as the “C” programming language or even assembly language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

The present invention may include a controller with a suction operation module and may also include a thermal or massage therapy module being an application program. The module(s) can be a stand-alone module or may also be incorporated into the operating system, the I/O device drivers or other such logical division of the data processing or control system.

The flowcharts and block diagrams of certain of the figures herein illustrate the architecture, functionality, and operation of possible implementations of suction collection or therapy means according to the present invention. In this regard, each block in the flow charts or block diagrams may represent a module, segment, or portion of code, which comprises one or more executable instructions for implementing the specified logical function(s). It should also be noted that in some alternative implementations, the functions noted in the blocks may occur out of the order noted in the figures. For example, two blocks shown in succession may in fact be executed substantially concurrently or the blocks may sometimes be executed in the reverse order, depending upon the functionality involved.

Thus, it will also be understood that one or more blocks of the block diagrams and combinations of blocks in block diagram figures can be implemented or directed to be carried out by computer program instructions. These computer program instructions may be loaded onto a computer or other programmable data processing apparatus to produce a machine, such that the instructions which execute on the computer or other programmable data processing apparatus create means for implementing the functions specified in the flowchart block or blocks. These computer program instructions may also be stored in a computer-readable memory that can direct a computer or other programmable data processing apparatus or associated hardware equipment to function in a particular manner diagrams.

The internal or in vivo collection capability with or without thermal or internal massage therapy provided by operations of the present invention can be carried out in a non-traumatic, minimally invasive manner. In certain embodiments, the heat generated during the thermal therapy can result in blood flow redistribution, which, in turn, may result in adhesion molecule difference and/or a difference in expression or prostate remodeling and collection of prostatic fluid or suctioning the fluid during the thermal therapy may enhance the therapeutic efficacy of the treatment. Suctioning fluid from the prostate during a thermal therapy provided by the instant invention may help regulate apoptosis in the prostate that may beneficially influence lower urinary tract symptoms in men with BPH or prostatitis. Further the therapy may act on the nerve endings in the inflamed prostate that may reduce the pain or improve the quality of life for the subject. Also, this may influence the formation of new blood vessels (angiogenisis) that may be considered a major contributor or of tissue development (particularly in BPH therapies).

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, where used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A method for obtaining a sample and/or treating a subject, comprising:
positioning an elongated transurethral catheter in the prostatic urethra of a subject, the catheter having a bladder anchoring balloon, at least one biosample entry port disposed axially away from the bladder anchoring balloon, and an axially extending biosample flow channel in fluid communication with the biosample entry port positioned internally in the catheter;
inflating the anchoring balloon to position the catheter so that the biosample entry port is proximate the prostatic or membraneous urethra of the subject; and
suctioning prostatic fluid from the prostatic urethra through the biosample entry port and into the biosample flow channel.

2. A method according to claim 1, further comprising directing the prostatic fluid to exit the body so that the suctioned prostatic fluid is substantially void of urine and then capturing the prostatic fluid after it exits the body.

3. A method according to claim 2, further comprising monitoring the quantity or flow rate of the captured fluid over a predetermined time.

4. A method according to claim 3, further comprising evaluating alterations in the level of at least one selected analyte in the captured prostatic fluid over time.

5. A method according to claim 1, wherein the catheter includes a urine drainage channel separate from the biosample flow channel, the method further comprising allowing urine to drain through the drainage channel during said suctioning step.

6. A method according to claim 1, wherein the catheter further comprises a treatment balloon disposed intermediate the at least one biosample entry port and the anchoring balloon, and wherein the method further comprises:
expanding the treatment balloon after the positioning step so that the expanded treatment balloon contacts prostatic tissue, wherein, in proper position, the expanded treatment balloon is sized and configured to reside in the prostatic urethra above the verumontanum; and
applying heat to the prostatic tissue based on said expanding step.

7. A method according to claim 6, wherein the suctioning step is carried out semi-continuously or continuously during the applying heat step.

8. A method according to claim 6, wherein the suctioning step is carried out at at least one desired time during the applying heating step.

9. A method according to claim 6, wherein the suctioning step is carried out before and after the applying heating step.

10. A method according to claim 6, wherein the suctioning step is carried out at selected intervals during the applying heat step.

11. A method according to claim 6, further comprising altering the pressure in the expanded treatment balloon such that the expanded treatment balloon repetitively laterally expands and contracts a desired distance in the prostatic urethra to internally massage the prostate.

12. A method according to claim 1, wherein the steps of applying heat and massage are carried out concurrently.

13. A method according to claim 12, wherein said internally massaging step is carried out by circulating pulsating heated fluid in an enclosed circulating travel path.

14. A method according to claim 6, wherein the subject is treated for prostatitis.

15. A method according to claim 6, wherein the subject is treated for BPH.

16. A method according to claim 6, wherein said applying heating step comprises heating fluid external of the body of the subject to a temperature of between about 40-67° C., and wherein said method further comprises directing circulating heated fluid to travel in a travel path in the catheter to and from the treatment balloon for a period of at least about 20 minutes.

17. A method according to claim 16, wherein the heated fluid circulating travel path is defined by a closed loop system which includes the catheter, an inlet conduit and an outlet conduit in fluid communication with the catheter, a heater, a pump, a pressure sensor and temperature sensors operably associated with the catheter, and wherein said massaging step comprises adding fluid to the closed loop system to increase the pressure in the treatment balloon at desired intervals over the duration of the treatment period.

18. A method for treating the prostate of a subject, comprising:
(a) inserting a catheter into the urethra of a subject, the catheter having, in serial order from the most distal portion, a bladder anchoring balloon, at least one expandable treatment balloon, and at least one fluid entry port formed in the wall of the catheter, the catheter also having an axially extending biosample flow path in fluid communication with the fluid entry port disposed internal of the catheter wall;
(b) expanding the bladder-anchoring balloon to contact and reside against the bladder neck of the subject to secure the catheter in position in the subject;
(c) heating fluid to a desired temperature;
(d) directing heated fluid to travel captured in the catheter to the at least one expandable treatment balloon;
(e) inflating the at least one treatment balloon responsive to the directing step, wherein, in position, the inflated treatment balloon takes on a radially expanded configuration and circumferentially contacts targeted tissue in the prostatic urethra;
(f) heating a targeted region in the prostatic urethra to a temperature of between about 40-67° C. for a desired treatment time of at least about 20 minutes; and
(g) collecting a biosample comprising prostatic fluid into the fluid entry port and into the biosample flow path of the catheter.

19. A method according to claim 18, wherein the biosample is substantially void of urine.

20. A method according to claim 18, wherein the collecting step comprises suctioning the biosample into the catheter.

21. A method according to claim 18, wherein the collecting step is carried out substantially continuously during the heating step.

22. A method according to claim 21, wherein the suctioning step is carried out at least one of before, after, and at least intermittently or at a plurality of discrete intervals for a predetermined period of time during the treatment.

23. A method according to claim 18, wherein the catheter further comprises a urine drainage channel in fluid isolation from the biosample flow path, and wherein urine is allowed to drain therein during the suctioning step.

24. A method according to claim 18, further comprising internally massaging a portion of the prostatic urethra by repetitively altering the fluid pressure in the treatment balloon causing the treatment balloon to repetitively expand and contract a desired distance in response thereto during the heating step.

25. A method according to claim 18, wherein said heating step heats the fluid to between about 40-45° C. to heat prostatic tissue for about 20-60 minutes.

26. A method according to claim 24, wherein said internally massaging step comprises circulating pulsating heated fluid in an enclosed circulating travel path in the catheter, the circulating fluid travel path being in fluid isolation from the biosample travel path.

27. A method according to claim 24, wherein the treatment balloon is repetitively expanded and contracted at a rate of about 1-5 times per second.

28. A method according to claim 24, wherein the expanded treatment balloon is sized and configured to reside in the prostatic urethra above the verumontanum during said heating and massaging steps.

29. A method according to claim 24, wherein the catheter comprises a blocking balloon disposed below the at least one entry port, the method further comprising:
expanding the blocking balloon so that it contacts tissue in the membraneous or bulbous urethra to define a treatment region extending above the expanded blocking balloon and below the anchoring balloon; and
delivering a medicament through the catheter and out the at least one entry port so that the medicament is injected into the treatment region proximate in time with the heating step.

30. A method according to claim 29, wherein the medicament is expelled from the catheter at elevated pressures.

31. A method of collecting a biosample in a subject, comprising:
inserting a catheter into the male urethra, the catheter having a biofluid travel path defined therein;
collecting a biosample from the prostatic urethra into the catheter in vivo; and
directing the biosample to travel in the biofluid travel path and to exit the body in a manner that maintains the biosample substantially void of urine.

32. A method according to claim 31, further comprising applying heat to the prostatic urethra proximate in time to or during the collecting step.

33. A method according to claim 31, further comprising allowing urine to drain from the bladder of the subject through the catheter during the collecting step without contacting the biosample in the biofluid travel path.

34. A method according to claim 31, further comprising massaging the prostatic urethra proximate in time or during the collecting step.

35. A method according to claim 34, wherein the catheter comprises an outwardly expandable balloon thereon, and wherein the massaging step is carried out internally by repetitively alternating the pressure in the balloon.

36. A method according to claim 31, wherein the collecting step comprises suctioning the biosample into the biofluid flow path of the catheter.

37. A method of collecting a biosample in a subject that is substantially void of urine, comprising:
inserting a catheter into a urethra of a subject, the catheter having a biofluid travel path defined therein;
suctioning a biosample from a targeted location along the urethra into the catheter in vivo; and
directing the suctioned biosample to travel in the biofluid travel path and to exit the body in a manner that maintains the biosample substantially void of urine.

38. A method according to claim 37, wherein the urethra is the female urethra.

39. A method according to claim 37, wherein the urethra is the male urethra.

40. A method according to claim 37, further comprising applying heat to a portion of the urethra proximate in time to or during the suctioning step.

41. A method according to claim 37, further comprising allowing urine to drain from the bladder of the subject during the suctioning step.

42. A method according to claim 37, further comprising massaging a portion of the urethra proximate in time or during the suctioning step.

43. A method according to claim 42, wherein the catheter comprises an outwardly expandable balloon thereon, and wherein the massaging step is carried out internally by repetitively alternating the pressure in the balloon.

44. A method according to claim 39, wherein the treatment is carried out proximate in time to administering radiation to the prostate of the subject.

45. A set of prostatic treatment catheters comprising:
a plurality of flexible elongated catheters sized and configured to be inserted into the male urethra, each catheter having an expandable treatment balloon positioned thereon and comprising a plurality of prostate drainage ports in communication with a prostate drainage lumen held internal of the catheter,
wherein the treatment balloons of the catheters in the set are configured with a length that is sized in the range of between about 1 cm to 6 cm such that, in operation, a selected treatment balloon resides above the verumontanum of the subject in the prostatic urethra.

46. A set of treatment catheters according to claim 45, wherein the catheter comprises a plurality of separate internal lumen fluid channels, including a circulating fluid inlet channel, a circulating fluid outlet channel, a urine drainage channel, and the prostate drainage lumen, and wherein the prostate drainage lumen is in fluid isolation from the other channels.

47. A system for collecting a prostatic fluid specimen in vivo in a subject, comprising:
a transurethral elongated catheter having an outer wall, the catheter having at least one prostate drainage port formed through the outer wall and an axially extending prostatic flow channel therein; and
a suction source in fluid communication with the at least one prostate drainage port, wherein in operation, the suction source provides a suction force sufficient to draw prostatic fluid into the at least one prostate drainage port and into the prostatic flow channel, thereby flowing out of the body of the subject so as to be able to be collected for evaluation.

48. A system for collecting a prostatic fluid specimen in vivo in a subject and/or facilitating the administration of a treatment to a subject, comprising:
an elongated transurethral catheter having an outer wall, the catheter comprising:
a plurality of axially extending internal fluid flow channels disposed in the catheter, an inlet circulating fluid channel, an outlet fluid circulating channel, a urinary drainage channel, and an axially extending prostatic fluid channel, wherein the prostatic fluid channel is in fluid isolation from the urine drainage and inlet and outlet channels;
an outwardly expandable treatment or dilatation balloon;
a bladder-anchoring balloon; and
at least one prostate drainage port formed through the outer wall of the catheter in fluid communication with the prostatic fluid channel;
a quantity of circulating fluid in the inlet and outlet channels;
a heater operably associated with fluid traveling in the inlet and outlet channels;
a pump operably associated with the circulating fluid to cause the fluid to circulate in the catheter;
at least one temperature sensor operably associated with the heater and the circulating fluid in the inlet and outlet channels; and
a suction source in fluid communication with the at least one prostate drainage port, wherein in operation, the suction source provides a suction force sufficient to draw prostatic fluid into the at least one prostate drainage port and into the prostatic flow channel, thereby flowing out of the body of the subject so as to be collected for evaluation.

49. A catheter or stent according to claim 48, wherein the suction source is the circulating fluid pump.

50. A transurethral catheter having an axially extending elongated body with an outer wall, the catheter comprising an anchoring balloon and at least one prostate drainage port formed through the outer wall and at least one axially extending prostate fluid collection channel located in the catheter in fluid communication with the at least one prostate drainage port, wherein, in proper position in the subject, the prostate drainage port is configured to reside proximate the verumontanum.

51. A catheter according to claim 50, wherein the at least one prostate drainage port is a plurality of drainage ports.

52. A catheter according to claim 50, wherein the anchoring balloon is a bladder-anchoring balloon, and wherein the catheter is devoid of a balloon located below the at least one prostate drainage port and above the urinary sphincter in the urethra of the subject.

53. A catheter or stent according to claim 50, wherein the anchoring balloon is a bladder anchoring balloon, and the catheter further comprises an outwardly expandable thermal treatment or dilation balloon located intermediate the anchoring balloon and the at least one prostate drainage port.

54. A computer program product for obtaining a biosample of the prostatic urethra, the computer program product comprising:
a computer readable storage medium having computer readable program code embodied in said medium, said computer-readable program code comprising:
computer readable program code computer readable program code for activating and applying a suction force to a fluid channel extending from a suction source located external of the body of the subject to the prostatic urethra via a catheter that is in fluid communication with the prostate; and
computer readable program code for drawing in and capturing a biosample comprising prostatic fluid in the catheter, the captured biosample being held so that it is substantially void of urine as it is directed to exit the subject in the catheter.

55. A computer program product for administering a thermal therapy to a subject, the thermal treatment being provided by a closed loop system having a heater, a circulating fluid pump, a suction source, and a trans-lumenal catheter configured and sized to be inserted through the urethra and having a biosample collection port and associated flow channel, and an outwardly expandable treatment balloon thereon that is configured, in operation, to expand while the catheter circulates heated fluid to heat a targeted region via the expandable treatment balloon, the computer program product comprising:
a computer readable storage medium having computer readable program code embodied in said medium, said computer-readable program code comprising:
computer readable program code for controlling the temperature of fluid circulating in the catheter so that the temperature of the fluid entering the catheter to travel to the expandable treatment balloon is between about 40-85° C.;
computer readable program code for timing the duration of the thermal massage treatment so that the treatment has a duration of between about 5 minutes to 1.5 hours; and
computer readable program code for activating the suction source to draw a biosample from the subject into the catheter and collect the sample so that it is delivered out of the subject.

56. A computer program product according to claim 55, wherein the targeted region is the prostatic and/or membraneous urethra, wherein the collected sample delivered out of the subject is in a condition that is representative of the concentration and content that it had when it was collected in the target region in the urethra, and wherein, the program product further comprising computer readable program code for internally massaging the prostatic urethra proximate in time to the capturing of the biosample by repetitively altering the expansion degree of the expandable treatment balloon by altering one of the pumping speed or pressure in the balloon.

57. A method for treating prostatitis, comprising:
(a) inserting a catheter with a prostatic fluid collection port and associated flow channel, a urinary drainage inlet and associated flow channel, and at least one expandable treatment balloon thereon into the urethra of a subject, the treatment balloon positioned to extend outwardly about the perimeter of a portion of the catheter;
(b) inflating the at least one treatment balloon so that, in position, the inflated treatment balloon takes on a radially expanded configuration and circumferentially contacts targeted tissue in the prostatic urethra;
(c) heating a targeted region in the prostatic urethra to a temperature of between about 40-47° C. for a desired treatment time of at least about 20 minutes thereby administering a thermal therapy to the prostate; and
(d) collecting prostatic fluid by directing the prostatic fluid into the collection port associated channel during or proximate in time to the treatment; and
(e) allowing urine to drain through the catheter drainage channel so that it is in fluid isolation from the collected prostatic fluid in the prostatic fluid collection channel.

58. A method according to claim 57, wherein the heating step is carried out by heating fluid to a desired temperature and directing the heated fluid such that it travels captured through the catheter to the at least one expandable treatment balloon in a circulating fluid travel path.

59. A method according to claim 57, wherein the collecting step comprises suctioning the prostatic fluid out at least once during the heating step.

60. A method according to claim 57, wherein the suctioning step is carried out substantially continuously during the heating step.

61. A method according to claim 57, further comprising internally massaging the prostatic urethra proximate in time or during the collecting step.

62. A method according to claim 61, further comprising and directing medicament to flow up into the catheter and out the prostatic fluid collection port after the massaging and heating steps to deliver the medicament to the targeted treatment region.

63. A method according to claim 57, wherein the catheter comprises a porous sleeve disposed over the treatment balloon, and the method further comprises delivering a medicament to the subject by allowing the medicament to travel through the membrane at a desired time before, during, or after the treatment.

64. A method for treating BPH, comprising:
(a) inserting a catheter with a suction port and associated flow channel and at least one expandable treatment balloon thereon into the urethra of a subject, the treatment balloon positioned to extend outwardly about the perimeter of a portion of the catheter;
(b) inflating the at least one treatment balloon, wherein, in position, the inflated treatment balloon takes on a radially expanded configuration and circumferentially contacts targeted tissue in the prostatic urethra;
(c) heating a targeted region in the prostatic urethra to a temperature of between about 40-67° C. for a desired treatment time of at least 20 minutes thereby administering a thermal therapy to the prostate; and
(d) collecting prostatic fluid in the catheter proximate in time to the treatment.

65. A method according to claim 64, wherein the heating step is carried out by heating fluid to a desired temperature and directing the heated fluid such that it travels captured through the catheter to the at least one expandable treatment balloon in a circulating fluid travel path, and wherein the method further comprises allowing urine to drain through the catheter so that it is in fluid isolation from the collected prostatic fluid.

66. A method according to claim 64, wherein the collecting step comprises suctioning the prostatic fluid into the catheter out at least once during or proximate in time but after the heating step.

67. A method according to claim 64, wherein the collecting step is carried out substantially continuously during the heating step.

68. A method according to claim 64, wherein said treatment is repeated in about 18 hours to about 1 month from the first treatment.

69. A method according to claim 65, wherein said heating step heats the fluid to about 45-65° C. to heat prostatic tissue for about 20-90 minutes.

70. A method according to claim 64, wherein the catheter includes a urinary drainage inlet and associated drainage channel, the drainage channel being separate from the prostatic fluid flow channel, and an anchoring balloon thereon.

71. A method according to claim 64, further comprising internally massaging the prostatic urethra proximate in time or during the collecting step.

72. A method according to claim 71, further comprising and directing the medicament to flow up into the catheter and out the prostatic fluid collection port after the massaging and heating steps to deliver the medicament to the targeted treatment region.

73. A method according to claim 64, wherein the catheter comprises a porous sleeve disposed over the treatment balloon, and the method further comprises delivering a medicament to the subject by allowing the medicament to travel through the membrane at a desired time before, during, or after the treatment.

74. A method according to claim 64, further comprising analyzing the collected prostatic fluid for one of quantity and constituent content.
